(19) United States
(12) Patent Application Publication
(10) Pub. No.: US 2023/0293483 A1
(43) Pub. Date: Sep. 21, 2023

(54) COMPOSITION FOR PREVENTING OR TREATING LIVER DISEASE, COMPRISING ICARITIN AND QUERCETIN

(71) Applicant: INDUSTRY-ACADEMIC COORPERATION FOUNDATION DAEGU HAANY UNIVERSITY, Gyeongsan-si, Gyeongsangbuk-do (KR)

(72) Inventors: Sang-Chan KIM, Daegu (KR); Sang-Mi PARK, Daegu (KR); ll-Je CHO, Daegu (KR); Jae-Kwang KIM, Daegu (KR); Eun-Ok KIM, Gyeongsan-si (KR); Chung-A PARK, Daegu (KR); Sung-Hui BYUN, Daegu (KR); Sook-Jahr PARK, Daegu (KR)

(73) Assignee: INDUSTRY-ACADEMIC COORPERATION FOUNDATION DAEGU HAANY UNIVERSITY, Gyeongsan-si, Gyeongsangbuk-do (KR)

(21) Appl. No.: 18/013,755

(22) PCT Filed: Sep. 25, 2020
(86) PCT No.: PCT/KR2020/013038

§ 371 Date: Dec. 29, 2022

(51) Int. Cl. A61K 31/353; A61P 1/16

(52) U.S. Cl. CPC A61K 31/353; A61P 1/16

(57) ABSTRACT

The present invention relates to a composition for preventing or treating liver disease, comprising icaritin and quercetin. It is ascertained that when icaritin and quercetin are used in combination rather than individually, the composition of the present invention has a superior effect of hepatic cell protection, and the optimal mixing ratio is derived, and it is also ascertained that the composition exhibits hepatoprotective action and does not induce toxicity in normal cells, and thus the present invention can be used as an active ingredient of a therapeutic agent for liver disease or a health functional food for preventing liver disease.

### TECHNICAL FIELD

The present invention relates to a composition including icaritin and quercetin for preventing or treating liver disease.

### BACKGROUND ART

The liver is an organ in which the in vivo metabolism is most active among the human body organs, and is located between the digestive system and the systemic circulatory system in the human body and performs the function of defending the whole body from ex vivo substances entering from the outside. Since ex vivo substances that enter the body first pass through the liver, the liver is at greater risk of being exposed to many toxic substances in addition to nutrients than other organs, so the probability of damage is also very high. However, the liver is an organ with excellent regenerative ability, and if there is slight damage, it may be sufficiently restored to normal. However, if the damage continues, a portion of the liver tissues is completely destroyed and the liver function is deteriorated, thus making it difficult to restore the damaged liver to the normal liver. When such liver damage becomes chronic, regardless of the cause, it progresses to liver fibrosis, cirrhosis, or liver cancer.

There are various causes of liver damage, such as stress-induced chronic fatigue, excessive intake of food or alcohol containing fat, viral infection, harmful substances such as various medicines, and lack of nutrition.

As a method for treating liver disease caused by liver damage, dietary therapy and pharmaceutical therapy are mainly used, and these two methods are used in combination. In addition, there are silymarin and biphenyl dimethyl dicarboxylate (BDD) as representative therapeutic agents for liver disease, but these drugs are not fundamental therapeutic agents, either. Therapeutic agents with other developed compounds may cause various side effects in the body, and thus are not being commercialized.

Accordingly, there is an urgent need to develop new therapeutic agents for liver disease using natural substances, which have excellent therapeutic effects enough to replace conventional therapeutic agents and have no side effects even when administered in large quantities or for a long period of time.

In this regard, the present inventors evaluated the hepatoprotective effect against oxidative stress when used in combination compared to the use of icaritin or quercetin alone, evaluated the hepatoprotective effect of the composition including icaritin and quercetin of an embodiment of the present invention, and confirmed that the composition including icaritin and quercetin might be used as materials for the treatment and prevention of liver disease, and then completed the present invention.

### DISCLOSURE

### Technical Problem

An aspect of the present invention is to provide a pharmaceutical composition for preventing or treating liver disease.

Another aspect of the present invention is to provide a health functional food composition for preventing or alleviating liver disease.

Yet another aspect of the present invention is to provide a health food composition for preventing or alleviating liver disease.

### Technical Solution

An embodiment of the present invention provides a pharmaceutical composition including icaritin and quercetin for preventing or treating liver disease.

In addition, an embodiment of the present invention provides a health functional food composition including icaritin and quercetin for preventing or alleviating liver disease.

Furthermore, an embodiment of the present invention provides a health food composition including icaritin and quercetin for preventing or alleviating liver disease.

### Advantageous Effects

The present invention relates to a composition including icaritin and quercetin for preventing or treating liver disease. It is ascertained that when icaritin and quercetin are used in combination rather than individually, the composition of the present invention has a superior effect of hepatic cell protection, and the optimal mixing ratio is derived, and it is also ascertained that the composition exhibits hepatoprotective action and does not induce toxicity in normal cells, and thus the present invention can be used as an active ingredient of a therapeutic agent for liver disease or a health functional food for preventing liver disease.

### DESCRIPTION OF DRAWINGS

FIG. 1 shows the results of measuring the cell viability of icaritin, quercetin, genistein, and luteolin against oxidative stress induced by AA+iron.

FIG. 2 shows the results of measuring the cell viability of the mixed use of icaritin and quercetin against oxidative stress induced by AA+iron.

FIG. 3 shows the synergistic effect on the inhibition of apoptosis according to the mixed use of icaritin and quercetin in oxidative stress induced by AA+iron.

FIG. 4 shows the cell viability according to the mixing ratio of icaritin and quercetin.

FIG. 5 is a result of comparing the cell viability between icaritin 1.25 μM+quercetin 6.25 μM and silymarin-based drugs.

FIG. 6 is a result of measuring ALT and AST, which are indicators of liver damage, as a result of measuring the effects of icaritin and quercetin on CCl₄-induced liver damage animal models.

### BEST MODES OF THE INVENTION

Hereinafter, the present invention will be described in detail.

Pharmaceutical Composition for Preventing or Treating Liver Disease

An embodiment of the present invention provides a pharmaceutical composition including icaritin and quercetin for preventing or treating liver disease.

In an embodiment of the present invention, the liver disease is liver cirrhosis, liver fibrosis, acute hepatitis, chronic hepatitis, or liver cancer.

In an embodiment of the present invention, the icaritin and the quercetin include 0.5 to 10 parts by weight of quercetin based on 1 part by weight of icaritin, preferably 0.5 to 6 parts by weight of quercetin based on 1 part by weight of icaritin.

According to an embodiment of the present invention, the composition including icaritin and quercetin of the present invention has no cytotoxicity and has an excellent hepatocellular protective effect against oxidative stress (see Experimental Example 1).

For reference, it is known that an increase in intracellular arachidonic acid (AA) opens the mitochondrial permeability transition pore (mPTP) to increase the release of cytochrome-c into the cytosol, induces apoptosis, and also increases the concentration of ceramide, so that the cytotoxicity of arachidonic acid (AA) occurs. After AA treatment, treatment with iron catalyzes oxidation, increases cellular oxidative stress, and causes mitochondrial dysfunction.

In order to identify the hepatocellular protective effect of the composition including icaritin and quercetin of an embodiment of the present invention, HepG2 cells, a human-derived liver parenchymal cell line, were treated with arachidonic acid (AA), and then treated with iron to induce the oxidative stress of the cells (named ‘AA+iron’). As a result, the hepatocellular protective effect through mitochondrial protective action was excellent, and it was shown to have significant hepatoprotective action also in animal models (see Experimental Example 2).

According to an embodiment of the present invention, it was identified through the liver damage evaluation indicators ALT and AST that the composition including icaritin and quercetin of an embodiment of the present invention was effective for hepatotoxicity induced by intraperitoneal administration of CCl₄, and it was identified that there was a protective effect against liver damage through a significant decrease in ALT and AST values according to the treatment of the composition including icaritin and quercetin (see Experimental Example 3).

For reference, ALT sensitively reflects hepatocellular degeneration and necrosis among organs, and an increase in ALT most meaningfully reflects the presence of liver damage. Accordingly, ALT is an ideal indicator to evaluate liver damage, and has been reported to have very high sensitivity, specificity, positive and negative predictive values for liver damage.

AST, together with ALT, is present in a large amount in myocardium, liver, skeletal muscle, kidney, etc., and is present in extremely small amounts in the blood. Accordingly, elevated levels of AST and ALT in the blood reflect degeneration and necrosis of cells in the organs in which they are distributed, and are widely used particularly as indicators of liver and heart diseases.

Health Functional Food or Health Food Composition for Preventing or Alleviating Liver Disease

An embodiment of the present invention provides a health functional food or health food composition including icaritin and quercetin for preventing or alleviating liver disease.

In an embodiment of the present invention, the icaritin and quercetin include 0.5 to 10 parts by weight of quercetin based on 1 part by weight of icaritin, preferably 0.5 to 6 parts by weight of quercetin based on 1 part by weight of icaritin.

When the icaritin and quercetin of an embodiment of the present invention are used as a health functional food and a health food composition, there is no particular limitation on the type of food. Examples of foods to which the icaritin and quercetin of an embodiment of the present invention may be added include drinks, meats, sausages, bread, biscuits, rice cakes, chocolate, candies, snacks, confectioneries, pizza, instant noodles, other noodles, gums, dairy products including ice cream, various soups, drinking water, alcoholic beverages, vitamin complexes, milk products, processed milk products, and the like, and include all health functional foods and health food compositions in a typical sense.

The health functional food and the health food composition including icaritin and quercetin according to an embodiment of the present invention may be added to food as it is or may be used together with other foods or food ingredients, and may be appropriately used according to a typical method. The mixing amount of icaritin and quercetin may be suitably determined depending on the purpose of use (for prevention or alleviation). In general, the amount of the composition in the health functional food and health food composition may be added to 0.1 to 90 parts by weight of the total food weight. However, in the case of long-term intake for the purpose of maintaining health or for the purpose of health control, the amount may be equal to or less than the above range, and icaritin and quercetin may also be used in an amount equal to or more than the above range because it poses no problem in terms of safety.

Other ingredients are not particularly limited, other than that the health functional food and health food composition of an embodiment of the present invention contains icaritin and quercetin of an embodiment of the present disclosure as an essential ingredient at an indicated ratio, and may contain various flavoring agents like those of a typical beverage, natural carbohydrates, and the like as additional ingredients. Examples of the aforementioned natural carbohydrates include typical sugars such as monosaccharides, for example, glucose, fructose and the like; disaccharides, for example, maltose, sucrose, and the like; and polysaccharides, for example, dextrin, cyclodextrin, and the like, and sugar alcohols such as xylitol, sorbitol, and erythritol. As flavoring agents in addition to those described above, a natural flavoring agent (thaumatin), a stevia extract (for example, rebaudioside A, glycyrrhizin, and the like), and a synthetic flavoring agent (saccharin, aspartame, and the like) may be advantageously used. The proportion of the natural carbohydrate is generally about 1 to 20 g, and preferably about 5 to 12 g per 100 g of the health functional food and the health food composition of an embodiment of the present invention.

In addition to the above, the health function food and health food composition containing the icaritin and quercetin of an embodiment of the present invention may contain various nutrients, vitamins, minerals (electrolytes), flavoring agents such as synthetic flavoring agents and natural flavoring agents, colorants and thickening agents (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in carbonated beverages, or the like. In addition, the health functional food and the health food composition of an embodiment of the present invention may contain flesh for preparing natural fruit juice, fruit juice drinks, and vegetable drinks.

### Modes of the Invention

Hereinafter, the present invention will be described in more detail by examples. These examples are merely for explaining the present invention in more detail, and it will be apparent to those skilled in the art that the scope of the present invention is not limited to these examples.

<Reagent>

Anti-poly(ADP-ribose) polymerase (PARP), anti-caspase-3, horseradish peroxidase-conjugated goat anti-rabbit IgG, and horseradish peroxidase-conjugated goat anti-mouse IgG antibodies were purchased from Cell Signaling Technology (Beverly, MA, USA). Arachidonic acid (AA) was purchased from Calbiochem (San Diego, CA, USA), silybin was purchased from Tokyo Chemical Industry Co., Ltd. (Tokyo, Japan), and silychrisitn and silydianin were purchased from Wuhan ChemFaces Biochemical Co., Ltd. (Wuhan, China) and used. 3-(4,5-dimethylthiazol-2-yl)-2, 5-diphenyl-tetrazolium bromide (MTT), ferric nitrilotriacetic acid (Fe-NTA; iron), icaritin, quercetin, silymarin, genistein, luteolin, anti-β-actin antibodies, and other reagents were purchased from Sigma (St. Louis, MO, USA).

<Cell Culture and Treatment>

HepG2 cell, a human-derived liver parenchymal cell line, was purchased from the American Type Culture Collection (ATCC, Rockville, MD, USA). Dulbecco's modified Eagle's medium (DMEM; Gibco) mixed with heat-treated 10% fetal bovine serum (FBS; Gibco), 100 units/ml of penicillin, and 100 μg/mL of streptomycin (Gibco) was used to culture HepG2 cells in an incubator maintained at 37° C. and 5% CO₂ conditions. Cells were cultured in a 100 mm dish to a confluence of 80% or more, and subcultured twice a week at a ratio of 1:4. After serum-starvation for 12 hours, drugs were treated at different concentrations, and 1 hour later, 10 μM of AA was treated for 12 hours, and then treated with 5 μM of iron, and further cultured for 5 hours.

<Breeding of Laboratory Animals>

As experimental animals, 6-week-old male ICR mice (19-22 g) were supplied from Samtaco Bio Korea (Osan, Korea) and used in the experiment after being acclimated to the laboratory environment for one week. The breeding room environment maintained a light/dark cycle of 12-hour intervals at a temperature of 20-23° C., and a humidity of 50%, and feed (Nestle Purina Petcare Korea, Seoul, Korea) and drinking water were freely available. This study was conducted in compliance with all regulations after being approved by the Experimental Animal Ethics Committee (DHU IACUC) of Daegu Haany University (approval number: DHU2018-064).

<Treatment of Laboratory Animals>

In the experiment, the group without any treatment was designated as the Normal group, and the group in which hepatotoxicity was induced by intraperitoneal administration of CC14 0.5 mL/kg diluted 10% with corn oil was designated as the CCl₄ group. Each drug treatment group was orally administered for 4 days, and CCl₄ was intraperitoneally administered once 1 hour after the last administration. Experimental animals were sacrificed 24 hours after administration of CCl₄.

<Statistical Analysis>

Analysis values of all evaluations were expressed as mean±S.D. after 3 repetitions. Statistical significance between each group was analyzed by one-way analysis of variance, followed by post hoc testing by Tukey's honestly significant difference or Dunnett's T3 analysis depending on whether the assumption of equal variance was established. Statistical significance was set based on a P value of less than 0.05 or 0.01.

### <Experimental Example 1> Cell Viability Test (MTT Assay)

Cell viability was measured by MTT assay. HepG2 cells were dispensed in 0.5 mL/well at a concentration of 2×10⁵ cells/well in a 24-well plate, and then cultured for 24 hours. After serum was depleted for 12 hours, the drug was treated for 1 hour, and then 10 μM of AA was treated for 12 hours, and 5 μM of iron was added and cultured for 5 hours. Herein, 300 μL of MTT 0.5 mg/mL per well was treated and reacted at 37° C. for 2 hours. After carefully removing the medium and dissolving formazan crystals produced by reducing MTT with dimethylsulfoxide (DMSO), absorbance was measured at 570 nm using a microplate measuring instrument (Tecan Infinite M200 PRO, USA).

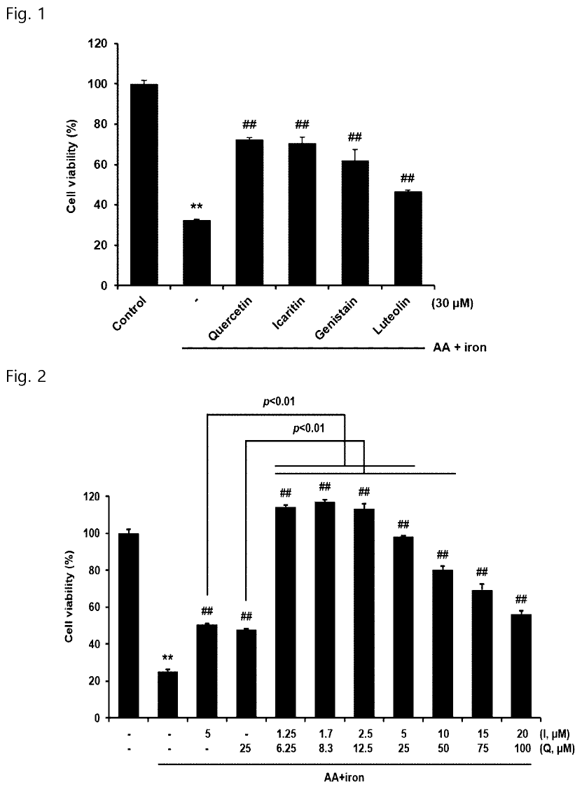

As a result of identifying the cell viability by inducing oxidative stress with AA+iron in HepG2 cells derived from hepatic parenchyma cells, it was identified that icaritin and quercetin exhibited the most significant hepatocellular protective effects at the same concentration (see FIG. 1).

In addition, in order to identify the cell viability according to the mixed use of icaritin and quercetin according to the results of FIG. 1, HepG2 cells were dispensed in 2×10⁵ cells/well into a 24-well plate and cultured for 24 hours. After treatment for 1 hour at the concentrations shown in Table 1 below, 10 μM of AA was treated for 12 hours, and 5 μM of iron was added and cultured for 5 hours. Herein, 3-(4,5-dimethylthiazol)-2,5-diphenyltetrazolium bromide (MTT) reagent was treated at 0.5 mg/mL per well, reacted at 37° C. for 2 hours, and then the formazan crystal produced was dissolved in DMSO. The absorbance was measured at 570 nm using a microplate measuring instrument (Tecan Infinite M200 PRO, USA).

TABLE 1

| Treatment | Comparative | Comparative | | | | | | | |
|---|---|---|---|---|---|---|---|---|---|
| concentration | Example 1 | Example 2 | Example 1 | Example 2 | Example 3 | Example 4 | Example 5 | Example 6 | Example 7 |
| Icaritin | 512241224 | — | 1.25 | 1.7 | 2.5 | 5 | 10 | 15 | 20 |
| (I, μM) | | | | | | | | | |
| Quercetin | — | 25 | 6.25 | 8.3 | 12.5 | 25 | 50 | 75 | 100 |
| (Q, μM) | | | | | | | | | |

As a result, as shown in FIG. 2, when icaritin 5 μM alone (Comparative Example 1) was used, the cell viability was about 50.8±0.4%, and when quercetin 25 μM alone (Comparative Example 2) was used, the cell viability was about 47.9±0.4%. In addition, rather than each treatment, when using a mixture of 5 μM of icaritin and 25 μM of quercetin (Example 5), an excellent cell viability of about 98.3±0.5% was shown, thus identifying that this represents a result value superior to the Colby formula predicted value of 73.5%.

In addition, when 1.25 μM of icaritin and 6.25 μM of quercetin were mixed and used (Example 1), when 1.7 μM of icaritin and 8.3 μM of quercetin were mixed and used (Example 2), or when 2.5 μM of icaritin and 12.5 μM of quercetin were mixed and used (Example 3), it was identified that the best cell viability was obtained.

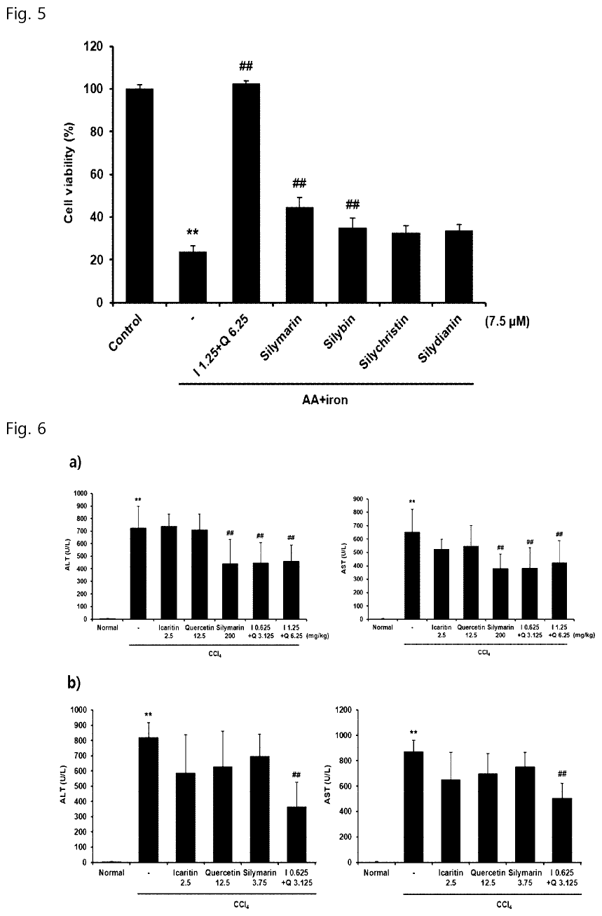

In addition, as a result of identifying the cell viability by inducing oxidative stress with AA+iron in HepG2 cells derived from hepatic parenchyma cells using 1.25 μM of icaritin and 6.25 μM of Quercetin, which are a combination of the lowest concentrations of the combination concentrations in Table 1 based on the results of FIG. 2, and using a silymarin complex (silymarin, silybin, silychristin, and silydianin), which is a representative liver disease therapeutic agent of the same concentration (7.5 μM), as shown in FIG. 5, it was identified that the cell viability was higher than that of silymarin, silybin, silycristin, and silydianin.

### <Experimental Example 2> Apoptosis Inhibitory Effect Induced by AA+Iron of Icaritin and Quercetin

The cultured and treated HepG2 cells were collected, and washed twice with phosphate buffered saline (PBS). Then, a lysis buffer mixed with radioimmunoprecipitation (RIPA) buffer (25 mM Tris-HCl pH 7.6, 150 mM NaCl, 1% NP-40, 1% sodium deoxycholate, 0.1% SDS) and Halt protease and phosphatase inhibitor cocktail (Thermo Fischer Scientific, Rockford, IL, USA) was added, reacted at 4° C. for 10 minutes, and centrifuged at 15,000×g for 30 minutes. Then, the supernatant was taken to prepare whole cell lysates. The protein content of the whole cell lysates was quantified using a BCA protein assay kit (Thermo). The extracted protein was mixed with Laemli's sample buffer, boiled for 5 minutes, and then electrophoresed on a 10% polyacrylamide gel. Proteins isolated by electrophoresis were transferred to a nitrocellulose membrane and blocked with 5% skim milk for 1 or more hours to suppress non-specific binding of antibodies. After reacting with the primary and secondary antibodies, the protein expression level was observed through an Imager 600 (Amersham Biosciences) using an enhanced chemiluminescent solution (Amersham Biosciences, Buckinghamshire, UK). Identification of the same amount of protein was verified through immunoblot analysis for β-actin, and the protein expression level was quantified using the ImageJ program (http://imagej.nih.gov/ij).

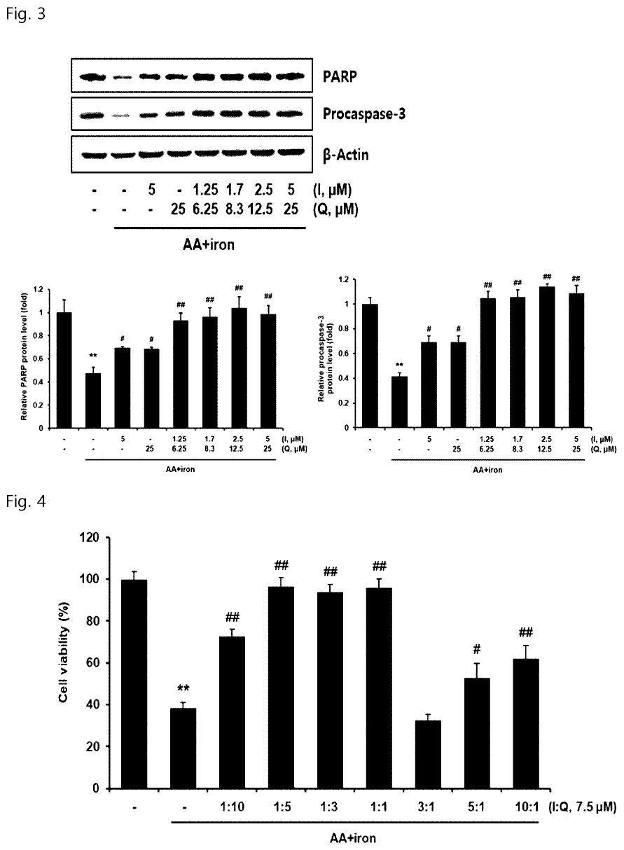

In order to evaluate whether the combined use of icaritin and quercetin may inhibit AA+iron-induced apoptosis, the expression of PARP and pro-caspase 3, which are anti-apoptotic markers among apoptosis-related proteins, was identified by immunoblot analysis. As a result, it was identified that the reduction of protein inhibited by AA+iron was significantly increased when icaritin and quercetin were used in combination than when icaritin or quercetin was used alone (see FIG. 3).

### <Experimental Example 3> Identification of Optimal Mixing Ratio of Icaritin and Quercetin

In order to derive the optimal mixing ratio of icaritin and quercetin, HepG2 cells were dispensed in a 24 well plate at 2×10⁵ cells/well and cultured for 24 hours, and then icaritin and quercetin at a concentration of 7.5 μM each were treated at various mixing ratios (icaritin:quercetin=1:10 to 10:1) for 1 hour. After treatment with 10 μM of AA for 12 hours, 5 μM of iron was added and cultured for 5 hours. Herein, 3-(4,5-dimethylthiazol)-2,5-diphenyltetrazolium bromide (MTT) reagent was treated at 0.1 mg/mL per well, reacted at 37° C. for 2 hours, and then the formazan crystal produced was dissolved in DMSO. The absorbance was measured at 570 nm using a microplate measuring instrument (Tecan Infinite M200 PRO, USA).

As a result, as shown in FIG. 4, it was identified that the mixing ratio of icaritin and quercetin of 1:5, 1:3, or 1:1 showed the best hepatoprotective effect.

### <Experimental Example 4> Effects of Icaritin and Quercetin on Liver Damage Induced by CCl₄

In order to identify the hepatoprotective effect of icaritin and quercetin of an embodiment of the present invention against damage caused by CCl₄, ALT and AST measurement experiments, which are used as ideal indicators for evaluating liver damage, were performed as follows.

After the experiment was completed, the experimental animals were anesthetized with CO₂, blood was collected from the abdominal vena cava, and the blood was centrifuged at 3,000×g and 4° C. for 15 minutes to obtain supernatant serum. ALT and AST in serum were analyzed using analysis kits (IVD Lab Co., Ltd., Uiwang, Korea) and an automated blood chemistry analyzer (Photometer 5010, Robert Riele GmbH & Co KG, Berlin, Germany).

As a result, as shown in FIG. 6, it was identified to show hematological values similar to the results of treatment with 200 mg/kg of silymarin at the mixed doses of icaritin and quercetin of 3.75 mg/kg and 7.5 mg/kg (see FIG. 6A). In addition, it was identified that the mixed use of icaritin and quercetin at the same dose of 3.75 mg/kg showed a significant hepatocellular protective effect, but silymarin had no significant effect (see FIG. 6B).

Accordingly, through the results of Experimental Examples 1 to 3, it was identified that the hepatocellular protective effect was significantly increased when the two components were mixed and used than when icaritin alone or quercetin alone was used. In addition, it was identified that a mixed use thereof showed a better effect at the same concentration than the representative therapeutic agent for liver disease, silymarin complex (silymarin, silybin, silychristin, and silydianin). Accordingly, it was identified that a similar effect was exhibited even when a lower concentration was used than the silymarin-based therapeutic agent used as a therapeutic agent for liver disease.

While the present invention has been particularly shown and described with reference to preferred embodiments thereof, it will be understood by those skilled in the art that various changes in form and details may be made therein without departing from the spirit and scope of the invention as defined by the appended claims. The embodiments should be considered in descriptive sense only and not for purposes of limitation. Therefore, the scope of the invention is defined not by the detailed description of the invention but by the appended claims, and all differences within the scope will be construed as being included in the present invention.

1. A pharmaceutical composition including icaritin and quercetin for preventing or treating liver disease.

2. The pharmaceutical composition of claim 1, wherein the liver disease is liver cirrhosis, liver fibrosis, acute hepatitis, chronic hepatitis, or liver cancer.

3. The pharmaceutical composition of claim 1, wherein the icaritin and the quercetin include 0.5 to 10 parts by weight of quercetin based on 1 part by weight of icaritin.

4. The pharmaceutical composition of claim 3, wherein the icaritin and the quercetin include 0.5 to 6 parts by weight of quercetin based on 1 part by weight of icaritin.

5. A health functional food composition including icaritin and quercetin for preventing or alleviating liver disease.

6. The health functional food composition of claim 5, wherein the health functional food is in the form of tablets, capsules, pills, or liquids.

7. The health functional food composition of claim 5, wherein the icaritin and the quercetin include 0.5 to 10 parts by weight of quercetin based on 1 part by weight of icaritin.

8. The health functional food composition of claim 7, wherein the icaritin and the quercetin include 0.5 to 6 parts by weight of quercetin based on 1 part by weight of icaritin.

9. A health food composition including icaritin and quercetin for preventing or alleviating liver disease.

10. The health food composition of claim 9, wherein the health food is selected from the group consisting of various drinks, meats, sausages, bread, candies, snacks, instant noodles, ice cream, milk products, soups, sports drinks, drinking water, alcoholic beverages, gums, teas, and vitamin complexes.
